(19) United States
(12) Patent Application Publication
(10) Pub. No.: US 2023/0380879 A1
(43) Pub. Date: Nov. 30, 2023

(54) END EFFECTOR ASSEMBLY WITH THERMAL CUTTING ELEMENT

(71) Applicant: Covidien LP, Mansfield, MA (US)

(72) Inventors: James D. Allen, IV, Broomfield, CO (US); William E. Robinson, Boulder, CO (US); Daniel A. Joseph, Golden, CO (US); John A. Hammerland, III, Arvada, CO (US); Kenneth E. Netzel, Loveland, CO (US)

(21) Appl. No.: 18/027,682

(22) PCT Filed: Aug. 25, 2021
(86) PCT No.: PCT/US21/47477

§ 371 Date: Mar. 22, 2023

Related U.S. Application Data
(60) Provisional application No. 63/082,582, filed on Sep. 24, 2020.

(51) Int. Cl. A61B 18/08; A61B 18/14

(52) U.S. Cl. CPC A61B 18/085; A61B 18/1445; A61B 2018/00077

(57) ABSTRACT

An end effector assembly for an electrosurgical instrument includes a pair of opposing jaw members each having a jaw housing supporting an electrically conductive tissue engaging surface thereon. The tissue engaging surfaces are disposed in opposition relative to one another and one or both of the jaw members are movable relative to one another to grasp tissue therebetween. First, second and third thermal cutting elements are disposed on one or both of the tissue engaging surfaces. Each thermal cutting element is independently activatable (or activatable in pairs) relative to the tissue engaging surfaces and each thermal cutting element connects to an electrosurgical energy source. The first thermal cutting element is exposed along the length of the tissue engaging surface, the second thermal cutting element surrounds the distal tip of the jaw member and the third thermal cutting element is disposed on the back of the jaw housing.

### FIELD

The present disclosure relates to surgical instruments and, more particularly, to thermal cutting elements, electrosurgical instruments including thermal cutting elements, and methods of manufacturing thermal cutting elements.

### BACKGROUND

A surgical forceps is a pliers-like instrument that relies on mechanical action between its jaw members to grasp, clamp, and constrict tissue. Electrosurgical forceps utilize both mechanical clamping action and energy to heat tissue to treat, e.g., coagulate, cauterize, or seal, tissue. Typically, once tissue is treated, the surgeon has to accurately sever the treated tissue. Accordingly, many electrosurgical forceps are designed to incorporate a knife that is advanced between the jaw members to cut the treated tissue. As an alternative to a mechanical knife, an energy-based tissue cutting element may be provided to cut the treated tissue using energy, e.g., thermal, electrosurgical, ultrasonic, light, or other suitable energy.

### SUMMARY

As used herein, the term “distal” refers to the portion that is being described which is further from a user, while the term “proximal” refers to the portion that is being described which is closer to a user. Further, to the extent consistent, any or all of the aspects detailed herein may be used in conjunction with any or all of the other aspects detailed herein.

Provided in accordance with aspects of the present disclosure is an end effector for a surgical instrument that includes a pair of opposing jaw members each having a jaw housing supporting an electrically conductive tissue engaging surface thereon disposed in opposition relative to one another. One or both jaw members are movable relative to the one other to grasp tissue therebetween. The electrically conductive tissue engaging surfaces are adapted to connect to an electrosurgical energy source.

A first thermal cutting element is disposed on one or both of the electrically conductive tissue engaging surfaces, the first thermal cutting element independently activatable relative to the electrically conductive tissue engaging surfaces and adapted to connect to an energy source. The first thermal cutting element is exposed along the length of the electrically conductive tissue engaging surface. A second thermal cutting element is disposed at a distal end of the jaw member, the second thermal cutting element covering a substantial portion of the distal end thereof. The second thermal cutting element is independently activatable relative to the electrically conductive tissue engaging surfaces and the first thermal cutting element and is adapted to connect to the energy source. A third thermal cutting element is disposed at a distal end of the jaw member, the third thermal cutting element covering a portion of the distal end of the jaw member and extending proximally therefrom. The third thermal cutting element is independently activatable relative to the electrically conductive tissue engaging surfaces and the first and second thermal cutting elements and is adapted to connect to the energy source. In aspects according to the present disclosure, the cutting element is electrically conductive.

In aspects according to the present disclosure, the thermal cutting element may be a single component with three different circuits to operate there different heating zones.

In aspects according to the present disclosure, a gap is defined between the first and second thermal cutting elements to mitigate thermal exchange therebetween. In other aspects according to the present disclosure, a gap is defined between the second and third thermal cutting elements to mitigate thermal exchange therebetween.

In aspects according to the present disclosure, the first thermal cutting element is activatable along with the first and second sealing surfaces to enhance tissue sealing. In other aspects according to the present disclosure, the first and second thermal cutting elements are activatable along with the first and second sealing surfaces to maximize tissue sealing.

In aspects according to the present disclosure, the second and third thermal cutting elements are activatable to maximize tissue scoring. In other aspects according to the present disclosure, the second or third thermal cutting elements are activatable to coagulate, blanch, dissect or score tissue.

In aspects according to the present disclosure, one or both of the first and second thermal cutting elements is activatable to cut tissue disposed between the jaw members upon activation thereof.

In aspects according to the present disclosure, the second thermal cutting element is configured to dissect or score tissue upon activation and distal movement along tissue. In other aspects according to the present disclosure, the third thermal cutting element is configured to dissect or score tissue upon activation and proximal movement along tissue.

Provided in accordance with aspects of the present disclosure is another embodiment of an end effector for a surgical instrument that includes a pair of opposing jaw members each having a jaw housing supporting an electrically conductive tissue engaging surface thereon disposed in opposition relative to one another. One or both jaw members are movable relative to the one other to grasp tissue therebetween. The electrically conductive tissue engaging surfaces are adapted to connect to an electrosurgical energy source. A cutting element is disposed on one or both of the electrically conductive tissue engaging surfaces. The cutting element is independently activatable relative to the electrically conductive tissue engaging surfaces and is adapted to connect to an energy source. The cutting element includes a conductive, corrugated wire extending along a substantial length of the electrically conductive tissue engaging surface(s). In aspects according to the present disclosure, the cutting element is electrically conductive.

In aspects according to the present disclosure, the conductive, corrugated wire includes an exposed edge having serrations therealong configured to facilitate cutting of tissue upon activation thereof. In other aspects according to the present disclosure, the serrations are configured to induce areas of high heat concentration upon activation of the conductive, corrugated wire enhancing tissue division.

Provided in accordance with aspects of the present disclosure is another embodiment of an end effector for a surgical instrument that includes a pair of opposing jaw members each having a jaw housing supporting an electrically conductive tissue engaging surface thereon disposed in opposition relative to one another. One or both jaw members are movable relative to the one other to grasp tissue therebetween. The electrically conductive tissue engaging surfaces are adapted to connect to an electrosurgical energy source. A thermal cutting element is disposed on one or both of the electrically conductive tissue engaging surfaces, the thermal cutting element is independently activatable relative to the electrically conductive tissue engaging surfaces and is adapted to connect to an energy source. The thermal cutting element includes a corrugated wire extending along a substantial length of the electrically conductive tissue engaging surface. In aspects according to the present disclosure, the cutting element is electrically conductive.

In aspects according to the present disclosure, the corrugated wire includes an exposed edge having serrations therealong configured to facilitate cutting of tissue upon heating thereof. In other aspects according to the present disclosure, the serrations are configured to induce areas of high heat concentration upon activation of the corrugated wire enhancing tissue division.

### BRIEF DESCRIPTION OF DRAWINGS

The above and other aspects and features of the present disclosure will become more apparent in view of the following detailed description when taken in conjunction with the accompanying drawings wherein like reference numerals identify similar or identical elements.

FIG. 1 is a perspective view of a shaft-based electrosurgical forceps provided in accordance with the present disclosure shown connected to an electrosurgical generator;

FIG. 2 is a perspective view of a hemostat-style electrosurgical forceps provided in accordance with the present disclosure;

FIG. 3 is a schematic illustration of a robotic surgical instrument provided in accordance with the present disclosure;

FIG. 4 is a perspective view of a distal end portion of the forceps of FIG. 1, wherein first and second jaw members of an end effector assembly of the forceps are disposed in a spaced-apart position;

FIG. 5A is a bottom, perspective view of the first jaw member of the end effector assembly of FIG. 4;

FIG. 5B is a top, perspective view of the second jaw member of the end effector assembly of FIG. 4;

FIG. 6 is a schematic, side view of an end effector including one embodiment of a thermal cutting element according to the present disclosure; and

FIG. 7 is a schematic, top view of an end effector including another embodiment of a thermal cutting element according to the present disclosure.

### DETAILED DESCRIPTION

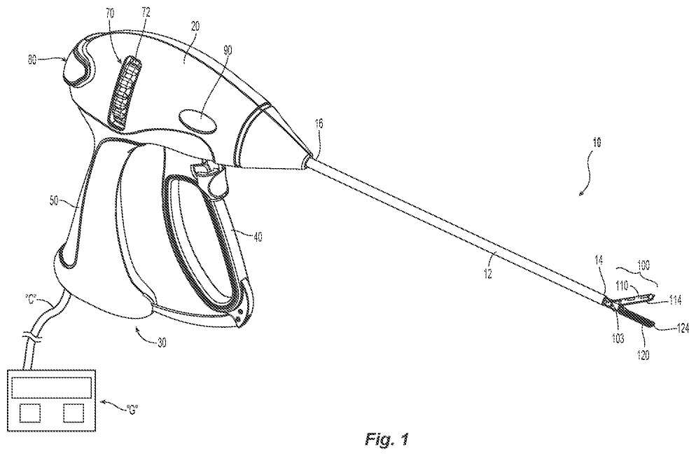

Referring to FIG. 1, a shaft-based electrosurgical forceps provided in accordance with the present disclosure is shown generally identified by reference numeral 10. Aspects and features of forceps 10 not germane to the understanding of the present disclosure are omitted to avoid obscuring the aspects and features of the present disclosure in unnecessary detail.

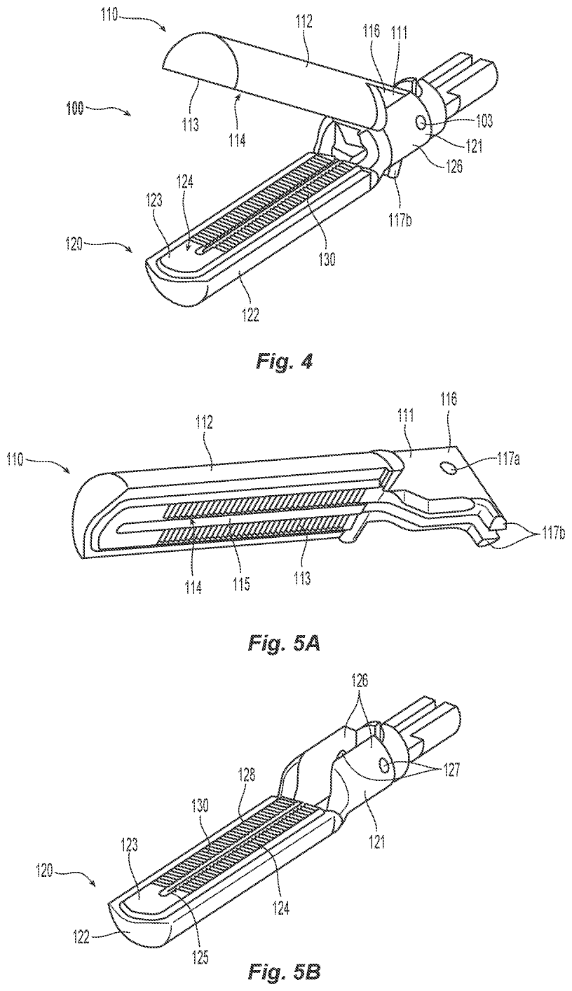

Forceps 10 includes a housing 20, a handle assembly 30, a trigger assembly 60, a rotating assembly 70, a first activation switch 80, a second activation switch 90, and an end effector assembly 100. Forceps 10 further includes a shaft 12 having a distal end portion 14 configured to (directly or indirectly) engage end effector assembly 100 and a proximal end portion 16 that (directly or indirectly) engages housing 20. Forceps 10 also includes cable “C” that connects forceps 10 to an energy source, e.g., an electrosurgical generator “G.” Cable “C” includes a wire (or wires) (not shown) extending therethrough that has sufficient length to extend through shaft 12 in order to connect to one or both tissue-treating surfaces 114, 124 of jaw members 110, 120, respectively, of end effector assembly 100 (see FIG. 4) to provide energy thereto. First activation switch 80 is coupled to tissue-treating surfaces 114, 124 (FIG. 4) and the electrosurgical generator “G” for enabling the selective activation of the supply of energy to jaw members 110, 120 for treating, e.g., cauterizing, coagulating/desiccating, and/or sealing, tissue. Second activation switch 90 is coupled to thermal cutting element 130 of jaw member 120 (FIG. 4) and the electrosurgical generator “G” for enabling the selective activation of the supply of energy to thermal cutting element 150 for thermally cutting tissue.

Alternatively, sealing and cutting may be activated by a single switch 80, 280. For example, after the seal cycle is completed, and if the surgeon is still activating the switch 80, 280, the generator “G” would then activate the cut cycle automatically. If the surgeon lets off the switch 80, 280, all energy delivery modalities immediately cease. The generator “G” may be configured to offer two different switchable modes for the surgeon enabling one device to have two modes of activation. For example, a first mode wherein the surgeon activates seal energy and cut energy with two separate switches, e.g., switch 80 and switch 90 (a double acting switch is also envisioned). Or a second mode which uses a single activation switch to accomplish both sealing and cutting functions controlled by the generator, feedback or an algorithm. Switch 80, 280 may be located on the housing 20, the handle 40 or the knife trigger.

Handle assembly 30 of forceps 10 includes a fixed handle 50 and a movable handle 40. Fixed handle 50 is integrally associated with housing 20 and handle 40 is movable relative to fixed handle 50. Movable handle 40 of handle assembly 30 is operably coupled to a drive assembly (not shown) that, together, mechanically cooperate to impart movement of one or both of jaw members 110, 120 of end effector assembly 100 about a pivot 103 between a spaced-apart position and an approximated position to grasp tissue between tissue-treating surfaces 114, 124 of jaw members 110, 120. As shown in FIG. 1, movable handle 40 is initially spaced-apart from fixed handle 50 and, correspondingly, jaw members 110, 120 of end effector assembly 100 are disposed in the spaced-apart position. Movable handle 40 is depressible from this initial position to a depressed position corresponding to the approximated position of jaw members 110, 120. Rotating assembly 70 includes a rotation wheel 72 that is selectively rotatable in either direction to correspondingly rotate end effector assembly 100 relative to housing 20.

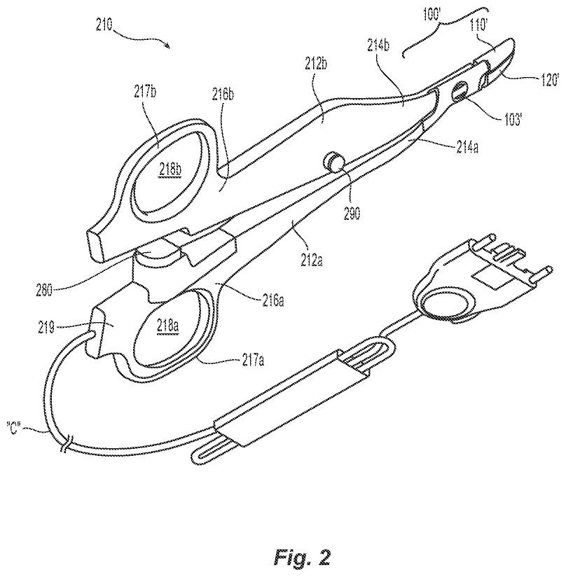

Referring to FIG. 2, a hemostat-style electrosurgical forceps provided in accordance with the present disclosure is shown generally identified by reference numeral 210. Aspects and features of forceps 210 not germane to the understanding of the present disclosure are omitted to avoid obscuring the aspects and features of the present disclosure in unnecessary detail.

Forceps 210 includes two elongated shaft members 212a, 212b, each having a proximal end portion 216a, 216b, and a distal end portion 214a, 214b, respectively. Forceps 210 is configured for use with an end effector assembly 100′ similar to end effector assembly 100 (FIG. 4). More specifically, end effector assembly 100′ includes first and second jaw members 110′, 120′ attached to respective distal end portions 214a, 214b of shaft members 212a, 212b. Jaw members 110′, 120′ are pivotably connected about a pivot 103′. Each shaft member 212a, 212b includes a handle 217a, 217b disposed at the proximal end portion 216a, 216b thereof. Each handle 217a, 217b defines a finger hole 218a, 218b therethrough for receiving a finger of the user. As can be appreciated, finger holes 218a, 218b facilitate movement of the shaft members 212a, 212b relative to one another to, in turn, pivot jaw members 110′, 120′ from the spaced-apart position, wherein jaw members 110′, 120′ are disposed in spaced relation relative to one another, to the approximated position, wherein jaw members 110′, 120′ cooperate to grasp tissue therebetween.

One of the shaft members 212a, 212b of forceps 210, e.g., shaft member 212b, includes a proximal shaft connector 219 configured to connect forceps 210 to a source of energy, e.g., electrosurgical generator “G” (FIG. 1). Proximal shaft connector 219 secures a cable “C” to forceps 210 such that the user may selectively supply energy to jaw members 110′, 120′ for treating tissue. More specifically, a first activation switch 280 is provided for supplying energy to jaw members 110′, 120′ to treat tissue upon sufficient approximation of shaft members 212a, 212b, e.g., upon activation of first activation switch 280 via shaft member 212a. A second activation switch 290 disposed on either or both of shaft members 212a, 212b is coupled to the thermal cutting element (not shown, similar to thermal cutting element 150 of jaw member 120 (FIG. 4)) of one of the jaw members 110′, 120′ of end effector assembly 100′ and to the electrosurgical generator “G” for enabling the selective activation of the supply of energy to the thermal cutting element for thermally cutting tissue.

Jaw members 110′, 120′ define a curved configuration wherein each jaw member is similarly curved laterally off of a longitudinal axis of end effector assembly 100′. However, other suitable curved configurations including curvature towards one of the jaw members 110, 120′ (and thus away from the other), multiple curves with the same plane, and/or multiple curves within different planes are also contemplated. Jaw members 110, 120 of end effector assembly 100 (FIG. 1) may likewise be curved according to any of the configurations noted above or in any other suitable manner.

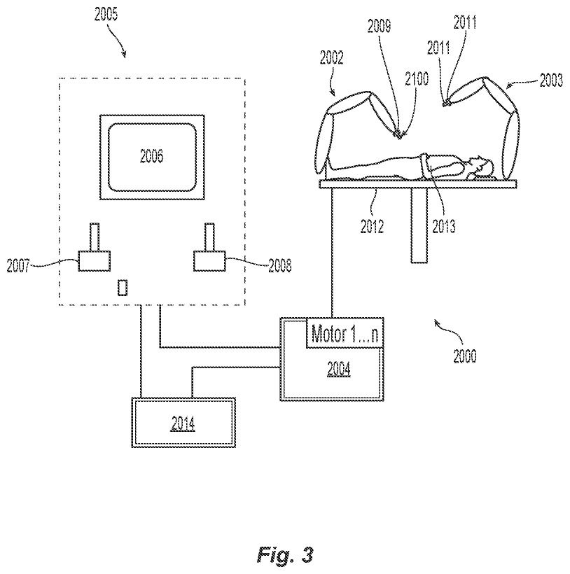

Referring to FIG. 3, a robotic surgical instrument provided in accordance with the present disclosure is shown generally identified by reference numeral 2000. Aspects and features of robotic surgical instrument 2000 not germane to the understanding of the present disclosure are omitted to avoid obscuring the aspects and features of the present disclosure in unnecessary detail.

Robotic surgical instrument 2000 includes a plurality of robot arms 2002, 2003; a control device 2004; and an operating console 2005 coupled with control device 2004. Operating console 2005 may include a display device 2006, which may be set up in particular to display three-dimensional images; and manual input devices 2007, 2008, by means of which a surgeon may be able to telemanipulate robot arms 2002, 2003 in a first operating mode. Robotic surgical instrument 2000 may be configured for use on a patient 2013 lying on a patient table 2012 to be treated in a minimally invasive manner. Robotic surgical instrument 2000 may further include a database 21014, in particular coupled to control device 2004, in which are stored, for example, pre-operative data from patient 2013 and/or anatomical atlases.

Each of the robot arms 2002, 2003 may include a plurality of members, which are connected through joints, and an attaching device 2009, 2011, to which may be attached, for example, an end effector assembly 2100, 2200, respectively. End effector assembly 2100 is similar to end effector assembly 100 (FIG. 4), although other suitable end effector assemblies for coupling to attaching device 2009 are also contemplated. End effector assembly 2200 may be any end effector assembly, e.g., an endoscopic camera, other surgical tool, etc. Robot arms 2002, 2003 and end effector assemblies 2100, 2200 may be driven by electric drives, e.g., motors, that are connected to control device 2004. Control device 2004 (e.g., a computer) may be configured to activate the motors, in particular by means of a computer program, in such a way that robot arms 2002, 2003, their attaching devices 2009, 2011, and end effector assemblies 2100, 2200 execute a desired movement and/or function according to a corresponding input from manual input devices 2007, 2008, respectively. Control device 2004 may also be configured in such a way that it regulates the movement of robot arms 2002, 2003 and/or of the motors.

Turning to FIG. 4, one embodiment of a known end effector assembly 100, as noted above, includes first and second jaw members 110, 120. Each jaw member 110, 120 may include a structural frame 111, 121, a jaw housing 112, 122, and a tissue-treating plate 113, 123 defining the respective tissue-treating surface 114, 124 thereof. Alternatively, only one of the jaw members, e.g., jaw member 120, may include the structural frame 121, jaw housing 122, and tissue-treating plate 123 defining the tissue-treating surface 124. In such embodiments, the other jaw member, e.g., jaw member 110, may be formed as a single unitary body, e.g., a piece of conductive material acting as the structural frame 111 and jaw housing 112 and defining the tissue-treating surface 114. An outer surface of the jaw housing 112, in such embodiments, may be at least partially coated with an insulative material or may remain exposed. For the purposes herein, the term “insulative” is defined to mean that the thermal or electrical conductivity of the feature is lower than the surrounding adjacent materials.

In embodiments, tissue-treating plates 113, 123 may be deposited onto jaw housings 112, 122 or jaw inserts (not shown) disposed within jaw housings 112, 122, e.g., via sputtering. Alternatively, tissue-treating plates 113, 123 may be pre-formed and engaged with jaw housings 112, 122 and/or jaw inserts (not shown) disposed within jaw housings 112, 122 via, for example, overmolding, adhesion, mechanical engagement, etc.

Referring in particular to FIGS. 4-5B, jaw member 110, as noted above, may be configured similarly as jaw member 120, may be formed as a single unitary body, or may be formed in any other suitable manner so as to define a structural frame 111 and a tissue-treating surface 114 opposing tissue-treating surface 124 of jaw member 120. Structural frame 111 includes a proximal flange portion 116 about which jaw member 110 is pivotably coupled to jaw member 120. In shaft-based or robotic embodiments, proximal flange portion 116 may further include an aperture 117a for receipt of pivot 103 and at least one protrusion 117b extending therefrom that is configured for receipt within an aperture defined within a drive sleeve of the drive assembly (not shown) such that translation of the drive sleeve, e.g., in response to actuation of movable handle 40 (FIG. 1) or a robotic drive, pivots jaw member 110 about pivot 103 and relative to jaw member 120 between the spaced-apart position and the approximated position. However, other suitable drive arrangements are also contemplated, e.g., using cam pins and cam slots, a screw-drive mechanism, etc.

Regardless of the particular configuration of jaw member 110, jaw member 110 may include a longitudinally-extending insulative member 115 extending along at least a portion of the length of tissue-treating surface 114. Insulative member 115 may be transversely centered on tissue-treating surface 114 or may be offset relative thereto. Further, insulative member 115 may be disposed, e.g., deposited, coated, etc., on tissue-treating surface 114, may be positioned within a channel or recess defined within tissue-treating surface 114, or may define any other suitable configuration. Additionally, insulative member 115 may be substantially (within manufacturing, material, and/or use tolerances) coplanar with tissue-treating surface 114, may protrude from tissue-treating surface 114, may be recessed relative to tissue-treating surface 114, or may include different portions that are coplanar, protruding, and/or recessed relative to tissue-treating surface 114. Insulative member 115 may be formed from, for example, ceramic, parylene, nylon, PTFE, polybenzimidazole, or other suitable material(s) (including combinations of insulative and non-insulative materials). Insulative member 115 may be disposed underneath the tissue treating surface 114 and be part of the jaw housing 112.

With reference to FIGS. 4 and 5B, as noted above, jaw member 120 includes a structural frame 121, a jaw housing 122, and a tissue-treating plate 123 defining the tissue-treating surface 124 thereof. Jaw member 120 further include a thermal cutting element 130. Structural frame 121 defines a proximal flange portion 126 and a distal body portion (not shown) extending distally from proximal flange portion 126. Proximal flange portion 126 is bifurcated to define a pair of spaced-apart proximal flange portion segments that receive proximal flange 111 of jaw member 110 therebetween and define aligned apertures 127 configured for receipt of pivot 103 therethrough to pivotably couple jaw members 110, 120 with one another.

Jaw housing 122 of jaw member 120 is disposed about the distal body portion of structural frame 121, e.g., via overmolding, adhesion, mechanical engagement, etc., and supports tissue-treating plate 123 thereon, e.g., via overmolding, adhesion, mechanical engagement, depositing (such as, for example, via sputtering), etc. Tissue-treating plate 123, as noted above, defines tissue-treating surface 124. A longitudinally-extending slot 125 is defined through tissue-treating plate 123 and is positioned to oppose insulative member 115 of jaw member 110 (FIG. 5A) in the approximated position. Slot 125 may extending through at least a portion of jaw housing 122, a jaw insert (if so provided), and/or other components of jaw member 120 to enable receipt of thermal cutting element 130 at least partially within slot 125.

Alternatively, in embodiments, the thermal cutting element 130 may be deposited atop the tissue-treating surface(s) 114, 124 in a strip-like manner. For example, a dielectric strip (not shown) is initially layered atop the flat or beveled slotless tissue-treating surface(s) 114, 124 or either jaw. A resistive thermal cutting element 130 is then layered atop the dielectric strip. A dielectric coating layer (not shown) is then applied over the thermal cutting element 130 to encapsulate the thermal cutting element 130 to contain unwanted heat or current leakage.

Thermal cutting element 130, more specifically, is disposed within longitudinally-extending slot 125 such that thermal cutting element 130 opposes insulative member 115 of jaw member 110 (FIG. 5A) in the approximated position. Thermal cutting element 130 may be configured to contact insulative member 115 (FIG. 5A) in the approximated position to regulate or contribute to regulation of a gap distance between tissue-treating surfaces 114, 124 in the approximated position. Alternatively or additionally, one or more stop members (not shown) associated with jaw member 110 and/or jaw member 120 may be provided to regulate the gap distance between tissue-treating surfaces 114, 124 in the approximated position.

Thermal cutting element 130 may be surrounded by an insulative member 128 disposed within slot 125 to electrically isolate thermal cutting element from tissue-treating plate 123. Alternatively or additionally, thermal cutting element 130 may include an insulative coating on at least the sides thereof for similar purposes. Thermal cutting element 130 and insulative member 128 may similarly or differently be substantially (within manufacturing, material, and/or use tolerances) coplanar with tissue-treating surface 124, may protrude from tissue-treating surface 124, may be recessed relative to tissue-treating surface 124, or may include different portions that are coplanar, protruding, and/or recessed relative to tissue-treating surface 124.

In embodiments where end effector assembly 100, or a portion thereof, is curved, longitudinally-extending slot 125 and thermal cutting element 130 may similarly be curved, e.g., wherein longitudinally-extending slot 125 and thermal cutting element 130 (or corresponding portions thereof) are relatively configured with reference to an arc (or arcs) of curvature rather than a longitudinal axis. Thus, the terms longitudinal, transverse, and the like as utilized herein are not limited to linear configurations, e.g., along linear axes, but apply equally to curved configurations, e.g., along arcs of curvature. In such curved configurations, insulating member 115 of jaw member 110 (FIG. 5A) is likewise curved.

Generally referring to FIGS. 1-5B, tissue-treating plates 113, 123 are formed from an electrically conductive material, e.g., for conducting electrical energy therebetween for treating tissue, although tissue-treating plates 113, 123 may alternatively be configured to conduct any suitable energy, e.g., thermal, microwave, light, ultrasonic, etc., through tissue grasped therebetween for energy-based tissue treatment. As mentioned above, tissue-treating plates 113, 123 are coupled to activation switch 80 and electrosurgical generator “G” (FIG. 1) such that energy may be selectively supplied to tissue-treating plates 113, 123 and conducted therebetween and through tissue disposed between jaw members 110, 120 to treat tissue, e.g., seal tissue on either side and extending across thermal cutting element 130.

Thermal cutting element 130, on the other hand, is configured to connect to electrosurgical generator “G” (FIG. 1) and second activation switch 90 to enable selective activation of the supply of energy to thermal cutting element 130 for heating thermal cutting element 130 to thermally cut tissue disposed between jaw members 110, 120, e.g., to cut the sealed tissue into first and second sealed tissue portions. Other configurations including multi-mode switches, other separate switches, etc. may alternatively be provided. Cross reference is made to U.S. Provisional Patent Application Ser. No. 62/952,232 the entire contents of which being incorporated by reference herein.

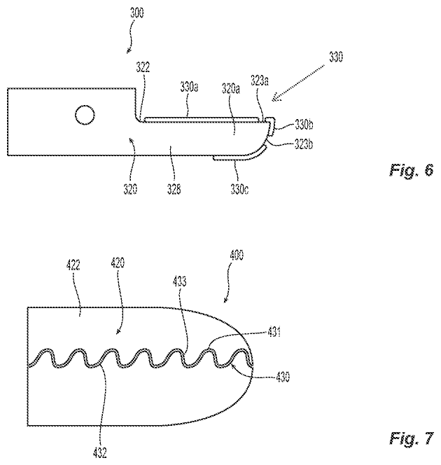

Referring to FIG. 6, another embodiment of a thermal cutting element 330 is shown as an assembly of thermal cutting elements for use with any of the above-described end effector assemblies and jaw configurations mentioned above. Thermal cutting element assembly 330 includes multiple heating elements, e.g., heating elements 330a-330c, disposed at various positions of the jaw member 320. A single component may be utilized with separate heating zones and circuits or multiple components may be used. In this instance, it is contemplated that thermally insulative materials or composites of materials with high thermal conductivity and low thermal conductivity may be used to isolate heat zones from one another instead of gaps therebetween.

Each heating element, e.g., heating element 330a, may be individually activated, simultaneously activated or sequentially activated with another heating element, heating element 330c, depending upon particular purposes or to achieve a particular result. Heating elements 330a-330c may be individually connected to a heating or energy source or work with a multiplexer to enable individual or paired activation thereof.

Heating element 330a is disposed along sealing surface 322 and extends substantially therealong. Heating element 330a may be centrally located along the sealing surface 322 or may be slightly askew depending upon a particular purpose. As mentioned above, heating element 330a may be individually connected to a generator or other heating source such that heating element 330a is independently activatable. Heating element 330a may work in unison with one or both of the other heating elements 330b, 330c to achieve a particular surgical result. Heating element 330b may also be activated simultaneously or sequentially to maximize sealing during the sealing process if energized with the sealing surface 322 and opposing sealing surface 113 (See FIGS. 1 and 4).

Heating element 330b is disposed proximate the distal end 320a of the jaw member 320 and may be configured to substantially cover the distal end 320a thereof. A gap 323a is defined between the distal-most end of heating element 330a and the proximal-most end of heating element 330b. Gap 323a is configured to dissipate heat emanating from heating element 330a during activation thereof giving the surgeon additional control of the various heatable areas of the jaw member 320. In other words, heat emanating from heating element 330a will not unintentionally thermally effect heating element 330b. Only when it is desirous for the surgeon to activate a heating source at the distal end of the jaw member 320, e.g., heating element 330b, will heat be generated at that particular area. Likewise, a gap 323b is defined between heating elements 330b and 330c to avoid unintended thermal exchange therebetween.

Instead of the surgeon having some degree or manual control over which hating element, e.g., heating element 330b, is activated, the generator “G” may be configured to make decisions based on one or more sensor inputs that may be utilized to determine surgeon intent. For example, if the instrument is configured with a tissue force sensor, then the generator “G” could be configured to activate the heating element 330b if particular conditions are met: the seal cycle completes, the switch 80, 280 is still being depressed, and/or force is being applied to the tissue grasped between the jaws members 110, 120 or 110′, 120′. As another example, if a jaw member 110 tip includes a capacitive touch sensor (e.g., at a distal end thereof) and a jaw positions sensor, the generator may be configured to activate the heating elements 330b or 330c if certain conditions are met: the jaw members 110, 120 are open, tissue is not sensed between the jaw members 110, 120 (e.g., via impedance), the capacitive touch sensor senses tissue in close proximity to the heating element 330b or 330c, and/or the surgeon is activating switch 80, 280.

As mentioned above, heating element 330b is configured to substantially cover the distal end 320a of jaw member 320 and may be activated either individually, sequentially or in unison with heating elements 330a and/or 330c depending upon a particular purpose or to achieve a particular surgical result. Heating element 330b may be used for maximizing sealing during the sealing process if energized with heating element 330a and the sealing surfaces 322, 113 (FIGS. 1 and 4). Moreover, heating element 330b may be used for tip sealing upon activation in combination with the sealing surfaces 322, 113 or may be used for blanching or spot coagulating tissue if energized or activated alone. Heating element 330b may also be utilized for scoring tissue when activated and moved distally or, in some cases, angled relative to the tissue and moved proximally (e.g., back-scoring).

Heating element 330c is disposed on the housing 328 toward the bottom of the distal end 320a and is configured to extend proximally therefrom along the back of jaw housing 328. As mentioned above, heating element 330c is separated from heating element 330b by gap 323b to avoid unintentional thermal exchange between heating elements 330b and 330c. As with the other heating elements 330a, 330b, heating element 330c may work in unison with one or both of the other heating elements 330a, 330b to achieve a particular surgical result. Heating element 330c may also be used for tissue scoring, e.g., back-scoring of tissue by dragging the jaw member 320 proximally across tissue. If energized with the heating element 330b, a surgeon can maximize tissue scoring. Moreover, the surgeon may energize heating element 330c to blanch or spot coagulate large tissue, e.g., larger tissue areas than heating element 330b can treat when activated alone.

Switch 90 may be utilized to activate the heating elements 330a-330c. Switch 90 may be configured in any fashion to enable individual activation of each heating element, e.g., heating element 330a, paired activation of two or more heating elements, e.g., 330a, 330b, and/or sequential activation of heating elements, e.g., 330a followed by 330b, followed by 330c, during a sealing and cutting cycle or for other surgical purposes, e.g., two-heater tissue scoring. Switch 90 may include three or more activation elements to accomplish this purpose or may include one or more toggles or rocker switches. A joystick-like switch 90 is also contemplated with different or varying activations of the various heating elements 330a-330c corresponding to the movement of the joystick-like switch 90. Other known switches are also contemplated.

In embodiments, the heating elements 330a-330c may be disposed on the same jaw member, e.g., jaw member 320, on opposing jaw members 110 (FIG. 1), 320 and combinations thereof.

Referring to FIG. 7, another embodiment of the thermal cutting element 430 is shown (in top view disposed along the sealing surface 422 of the jaw member 420) use with any of the above-described end effector assemblies and jaw configurations mentioned above. Thermal cutting element 430 includes a thermally conductive or electrically conductive wire 431 centrally disposed along the sealing surface 422. Thermal cutting element 430 may be centrally located along the sealing surface 422 or may be slightly askew depending upon a particular purpose. Conductive wire 431 is substantially corrugated or sinuous along the length of the sealing surface 422 which maximizes the thermal effect therealong, e.g., maximizes the power dissipation and power density due to increased length of an electrically conductive wire 431 or creates hot spots along a thermally conductive wire 431. The corrugations may be of any size or configuration including microtine to increase the overall surface area of the thermal cutting element 430.

The exposed edge 433 of the conductive wire 431 may include serrations 432 to mechanically assist cutting tissue. Moreover, the exposed serrations 432 along the edge 433 create points of high current or heat concentration (i.e., higher heat locations) at the edges 433 which, in turn, facilitates tissue division. The exposed serrated edge 433 of the conductive wire 431 may also assist cutting tissue in an energized/heat and pull-to-cut fashion, e.g., energizing or heating the conductive wire 431 while pulling the tissue between the jaw members 110 (FIG. 1), 420 away from the remaining tissue.

While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed is:

1. An end effector assembly for an electrosurgical instrument, comprising:
a pair of opposing jaw members each including a j aw housing supporting an electrically conductive tissue engaging surface thereon, the electrically conductive tissue engaging surfaces disposed in opposition relative to one another, at least one of the pair of jaw members movable relative to the other of the pair of jaw members to grasp tissue therebetween, the electrically conductive tissue engaging surfaces adapted to connect to an electrosurgical energy source; and
a first thermal cutting element disposed on at least one of the electrically conductive tissue engaging surfaces, the first thermal cutting element independently activatable relative to the electrically conductive tissue engaging surfaces and adapted to connect to an energy source, the first thermal cutting element exposed along the length of the at least one electrically conductive tissue engaging surface;
a second thermal cutting element disposed at a distal end of the at least one jaw member, the second thermal cutting element covering a substantial portion of the distal end of the at least one jaw member, the second thermal cutting element independently activatable relative to the electrically conductive tissue engaging surfaces and the first thermal cutting element and adapted to connect to the energy source; and
a third thermal cutting element disposed at a distal end of the at least one jaw member, the third thermal cutting element covering a portion of the distal end of the at least one jaw member and extending proximally therefrom, the third thermal cutting element independently activatable relative to the electrically conductive tissue engaging surfaces and the first and second thermal cutting elements and adapted to connect to the energy source.

2. The end effector assembly according to claim 1, wherein a gap is defined between the first and second thermal cutting elements to mitigate thermal exchange therebetween.

3. The end effector assembly according to claim 1, wherein a gap is defined between the second and third thermal cutting elements to mitigate thermal exchange therebetween.

4. The end effector assembly according to claim 1, wherein the first thermal cutting element is activatable along with the first and second sealing surfaces to enhance tissue sealing.

5. The end effector assembly according to claim 1, wherein the first and second thermal cutting elements are activatable along with the first and second sealing surfaces to maximize tissue sealing.

6. The end effector assembly according to claim 1, wherein the second and third thermal cutting elements are activatable to maximize tissue scoring.

7. The end effector assembly according to claim 1, wherein the second or third thermal cutting elements are activatable to coagulate, blanch, dissect or score tissue.

8. The end effector assembly according to claim 1, wherein at least one of the first or second thermal cutting elements is activatable to cut tissue disposed between the jaw members upon activation thereof.

9. The end effector assembly according to claim 1, wherein the second thermal cutting element is configured to dissect or score tissue upon activation and distal movement along tissue.

10. The end effector assembly according to claim 1, wherein the third thermal cutting element is configured to dissect or score tissue upon activation and proximal movement along tissue.

11. An end effector assembly for an electrosurgical instrument, comprising:
a pair of opposing jaw members each including a jaw housing supporting an electrically conductive tissue engaging surface thereon, the electrically conductive tissue engaging surfaces disposed in opposition relative to one another, at least one of the pair of j aw members movable relative to the other of the pair of jaw members to grasp tissue therebetween, the electrically conductive tissue engaging surfaces adapted to connect to an electrosurgical energy source; and
a cutting element disposed on at least one of the electrically conductive tissue engaging surfaces, the cutting element independently activatable relative to the electrically conductive tissue engaging surfaces and adapted to connect to an energy source, the cutting element including a conductive, corrugated wire extending along a substantial length of the at least one electrically conductive tissue engaging surface.

12. The end effector assembly according to claim 11, wherein the conductive, corrugated wire includes an exposed edge having serrations therealong configured to facilitate cutting of tissue upon activation thereof.

13. The end effector assembly according to claim 12, wherein the serrations are configured to induce areas of high heat concentration upon activation of the conductive, corrugated wire enhancing tissue division.

14. An end effector assembly for an electrosurgical instrument, comprising:
a pair of opposing jaw members each including a jaw housing supporting an electrically conductive tissue engaging surface thereon, the electrically conductive tissue engaging surfaces disposed in opposition relative to one another, at least one of the pair of jaw members movable relative to the other of the pair of jaw members to grasp tissue therebetween, the electrically conductive tissue engaging surfaces adapted to connect to an electrosurgical energy source; and
a thermal cutting element disposed on at least one of the electrically conductive tissue engaging surfaces, the thermal cutting element independently activatable relative to the electrically conductive tissue engaging surfaces and adapted to connect to an energy source, the thermal cutting element including a corrugated wire extending along a substantial length of the at least one electrically conductive tissue engaging surface.

15. The end effector assembly according to claim 14, wherein the corrugated wire includes an exposed edge having serrations therealong configured to facilitate cutting of tissue upon heating thereof.

16. The end effector assembly according to claim 15, wherein the serrations are configured to induce areas of high heat concentration upon activation of the corrugated wire enhancing tissue division.
